(19) United States
(12) Patent Application Publication
(10) Pub. No.: US 2024/0197354 A1
(43) Pub. Date: Jun. 20, 2024

(54) IRRIGATED ULTRASONIC DEVICE WITH FEATURES FOR DISSECTING DENSE TISSUE

(71) Applicant: Medtronic PS Medical, Inc., Fort Worth, TX (US)

(72) Inventors: Matthew S. COWLEY, Frederick, CO (US); David J. VAN TOL, Boulder, CO (US)

(21) Appl. No.: 18/086,505

(22) Filed: Dec. 21, 2022

Related U.S. Application Data
(63) Continuation-in-part of application No. 18/083,993, filed on Dec. 19, 2022, now abandoned.

(51) Int. Cl. A61B 17/32

(52) U.S. Cl. CPC A61B 17/320068; A61B 2017/00367; A61B 2017/320004; A61B 2017/32007; A61B 2017/320074; A61B 2017/320084

(57) ABSTRACT

Systems and methods whereby a surgical instrument includes an ultrasonic transducer contained in a housing and a transmission assembly at least partially received by the housing. The transmission assembly enabled to encapsulate a waveguide, wherein the waveguide transmits ultrasonic vibrations from the transducer to the ultrasonic blade. The ultrasonic blade includes a blade tip and at least one incorporated feature between a distal end of the ultrasonic blade and a proximal end, wherein the at least one incorporated feature includes one or more depressions or protrusions, creating abrasive surfaces and channels. The abrasive surfaces capable of breaking up tissue and the channels capable of collecting and expelling the broken-up tissue. A control switch operable to initiate and halt the ultrasonic transducer.

### CROSS-REFERENCE TO RELATED APPLICATIONS

This application is a continuation-in-part of U.S. application Ser. No. 18/083,993, filed Dec. 19, 2022, the disclosure of which is hereby incorporated by reference in its entirety.

### FIELD

The present technology is generally related to instruments or tools used in performing surgery on a patient and, more particularly, to surgical instruments with enhanced ultrasonic tissue dissection.

### BACKGROUND

Medical instruments utilized in performing orthopedic surgical procedures often use bone shavers, saws, and debridement devices to remove bone and other harder tissues from treatment areas. With the increasing demand for the development of minimally invasive surgical techniques, the difficulty of achieving satisfactory safety and accuracy of these procedures is increasing. For example, making even minor mistakes during any procedure may pose extremely dangerous risks, such as soft tissue and nerve damage that can have far-reaching and life-changing effects. Previously, surgeons mostly adopted medical instruments such as rongeurs, micro saws, high-speed grinding heads and the like to cut bone tissues, but the amount of bleeding can be substantial. Bone tissues are easy to damage themselves, as well as being surrounded by delicate soft tissue.

Currently, these medical instruments are being adapted to include the use of ultrasonics, such as ultrasonic dissectors or other cutting instruments. Ultrasonic dissectors function by sending a large amount of kinetic energy down a waveguide to a cutting accessory, such as a cutting blade with an ultrasonic tip. The blade uses kinetic energy and pressure to heat, fracture, or pulverize tissues. The pressure can be applied in the form of tissue tension or compression placed along the blade or to the tip such as making an enterotomy. Typically, these devices are of a rotary nature, but they can also be oscillating at sub-sonic or at ultrasonic velocities. In performing a cutting, shaving, or shaping operation, the cutting accessory will be exposed to varying amounts of force, creating stresses within the cutting accessory.

The use of an ultrasonic cutting instrument as a novel bone removal instrument has a number of advantages over conventional bone removal tools, including tissue selectivity, anti-roll-scraping properties, cold cutting, ease of operation, blood supply protection, and reduced procedure time. However, ultrasonic cutting instruments still present safety and effectiveness issues during the surgical procedure. In surgery, tissue density and elasticity properties can vary, and soft tissue avoids certain cutting risks due to partial absorption of high-frequency impact energy. For example, some tissue or ligaments are more easily cut during operations and, if surrounding the area in which the tougher tissues is being removed, can be negatively affected. Moreover, ultrasonic blades travel at an extremely high velocity and, thus, displacement must be accurate and manageable.

Therefore, there is a present need for improved ultrasonic technology, particularly to the efficiency and effectiveness of ultrasonic dissectors.

### SUMMARY

The technology is generally related to instruments or tools used in performing surgery on a patient and, more particularly, to surgical instruments with enhanced ultrasonic tissue dissection.

In one aspect, the present disclosure provides systems and methods whereby a surgical instrument includes an ultrasonic transducer contained in a housing and a transmission assembly at least partially received by the housing. The transmission assembly enabled to encapsulate a waveguide, wherein the waveguide transmits ultrasonic vibrations from the transducer to the ultrasonic blade. The ultrasonic blade includes a blade tip and at least one embedded feature between a distal end of the ultrasonic blade and a proximal end, wherein the at least one embedded feature includes one or more depressions and abrasive surfaces. The abrasive surfaces capable of breaking up tissue and the depressions capable of collecting the broken-up tissue. A control switch operable to initiate and halt the ultrasonic transducer.

In another aspect, the disclosure provides an ultrasonic surgical tip having an ultrasonic blade including a blade tip at a distal end couplable to a waveguide, encapsulated by a transmission assembly, at a proximal end. The waveguide is enabled to transmit ultrasonic vibrations from the transducer to the ultrasonic blade. The ultrasonic blade having at least one embedded feature, at least in part, stretching between the distal end and the proximal end. The at least one embedded feature includes one or more depressions and abrasive surfaces. The abrasive surfaces capable of breaking up tissue and the depressions capable of collecting the broken-up tissue.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

Subject matter hereof may be more completely understood in circumstances of the following detailed description of various embodiments in connection with the accompanying figures, in which:

FIG. 1 illustrates a cross-sectional view of an ultrasonic surgical instrument enabled to utilize an ultrasonic blade, according to embodiments.

FIG. 2 illustrates a perspective view of a dimpled pattern ultrasonic blade with a wedge-shaped blade tip, according to embodiments.

FIG. 3 illustrates a perspective view of a grooved pattern ultrasonic blade with an arc-shaped blade tip, according to embodiments.

FIG. 4 illustrates a perspective view of a grooved pattern ultrasonic blade, similarly depicted in FIG. 3, with a crenelated blade tip, according to embodiments.

FIG. 5 illustrates a perspective view of a grooved pattern ultrasonic blade, similarly depicted in FIG. 4, with dual lumen irrigation grooves, according to embodiments.

FIG. 6 illustrates a perspective view of a spirally grooved pattern ultrasonic blade, according to embodiments.

FIG. 7 illustrates a perspective view of a diamond grooved pattern ultrasonic blade, similarly depicted in FIG. 6, with a cross-shaped blade tip, according to embodiments.

FIG. 8 illustrates a perspective view of an ultrasonic blade, according to embodiments.

FIG. 9 illustrates a perspective view of a spiral pattern ultrasonic blade, according to embodiments.

FIG. 10A illustrates a side view of a scalpel-type ultrasonic blade, according to embodiments.

FIG. 10B illustrates a top view of the scalpel-type ultrasonic blade of FIG. 8A, according to embodiments.

While various embodiments are amenable to various modifications and alternative forms, specifics thereof have been shown by way of example in the drawings and will be described in detail. It should be understood, however, that the intention is not to limit the claimed disclosure to the particular embodiments described. On the contrary, the intention is to cover all modifications, equivalents, and alternatives falling within the spirit and scope of the subject matter as defined by the claims.

### DETAILED DESCRIPTION

Reference now will be made in detail to embodiments of the disclosure, one or more examples of which are illustrated in the drawings. Each example is provided by way of explanation in the disclosure and is not limited thereto. In fact, it will be apparent to those skilled in the art that various modifications and variations can be made in the present disclosure without departing from the scope of the disclosure. For instance, features illustrated or described as part of one embodiment can be used with another embodiment to yield a still further embodiment. Thus, it is intended that the present disclosure covers such modifications and variations as come within the scope of the appended claims and their equivalents. As used herein, the term “or” is generally intended to be inclusive (i.e., “A or B” is intended to mean “A or B or both”). The terms “first,” “second,” and “third” may be used interchangeably to distinguish one component from another and are not intended to signify location or importance of the individual components.

Throughout the specification, and in the claims, the term “connected” means a direct electrical, mechanical, or magnetic connection between the things that are connected, without any intermediary devices. The terms “coupled” or “integrated” mean either a direct electrical, mechanical, or magnetic connection between the things that are connected or an indirect connection through one or more passive or active intermediary devices. The term “circuit,” “module,” or “mechanism” may refer to one or more passive and/or active components that are arranged to cooperate with one another to provide a desired function.

The terms “substantially,” “close.” “approximately,” “near,” and “about” generally refer to being within +/−10% of a target value. Unless otherwise specified the use of the ordinal adjectives “first,” “second,” and “third,” etc., to describe a common object, merely indicate that different instances of like objects are being referred to and are not intended to imply that the objects so described must be in a given sequence, either temporally, spatially, in ranking, or in any other manner.

It is to be understood that the terms so used are interchangeable under appropriate circumstances such that the embodiments of the disclosure described herein are, for example, capable of operation in other orientations than those illustrated or otherwise described herein.

The terms “left,” “right.” “front,” “back,” “top,” “bottom,” “over,” “under,” and the like in the description and in the claims, if any, are used for descriptive purposes and not necessarily for describing permanent relative positions.

As described herein, embodiment of the present disclosure provides systems and methods related to instruments or tools used to perform surgery on a patient and, more particularly, to surgical instruments with enhanced ultrasonic tissue dissection.

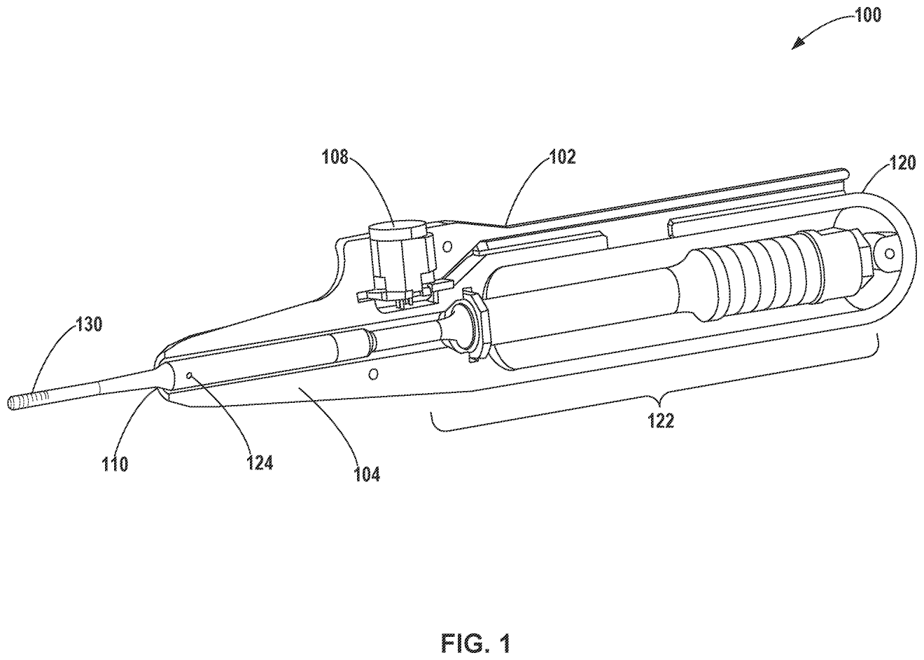

Referring to FIG. 1, FIG. 1 illustrates a cross-sectional view of an ultrasonic surgical instrument, according to embodiments. As will be described in greater detail below, surgical instrument 100 is operable to cut or drill through dense tissue, such as bone matter, using ultrasonic vibrational energy. In embodiments, the effect or motions may be similar to a jackhammer. In embodiments, the surgical instrument 100 may be an ultrasonic dissector, for example, a lead zirconate titanate (PZT) based ultrasonic dissector. In embodiments, surgical instrument 100 comprises a handle assembly 102, a transmission assembly 110, and an ultrasonic blade 130 in which may be elongated and circularly shaped. Handle assembly 102 comprises a housing 104 and one or more control switches 108. In embodiments, the housing 104, particularly through the transmission assembly 110 and the ultrasonic blade 130, may be solid, having no lumens or other hollow channels for aspiration. In embodiments, the handle assembly 102 and the ultrasonic blade 130 may have one or more lumens, which may provide irrigation or suction through the surgical instrument 100 substantially simultaneously during ultrasonic use, at least in part, through irrigation entry point 124 and corresponding lumens. In embodiments the use of saline, cooling fluids, or other similar fluids may be used as irrigates. In embodiments, the one or more lumens may be a single axial lumen each with an irrigation inlet enabling single directional flow. It should be understood, however, that various other suitable configurations may be used. %

In embodiments, an ultrasonic transducer assembly 120 may extend proximally from housing 104/hand assembly 102. Transducer assembly 120 may be coupled with a generator via a cable. It should also be understood that at least some of the functionality of a generator may be integrated into handle assembly 102, and that handle assembly 120 may even include a battery or other on-board power source such that a cable is omitted. It should be understood that transducer assembly 120 may receive electrical power from a generator or internal power source and convert that power into ultrasonic vibrations through piezoelectric principles. The generator or internal power source may thus include a power source and control module that is configured to provide a power profile to transducer assembly 120 that is particularly suited for the generation of ultrasonic vibrations through transducer assembly 120.

In embodiments, transducer assembly 120 may include an ultrasonic transducer 122 and an ultrasonic waveguide (not shown), encapsulated by the transmission assembly 110, which couples the ultrasonic transducer 122 with the ultrasonic blade 130. Ultrasonic transducer 122 may receive electrical power from power source, such as a generator, as described herein. By virtue of its piezoelectric properties, ultrasonic transducer 122 is operable to convert such electrical power into ultrasonic vibrational energy. Still other suitable ultrasonic vibrations may be utilized, as well as various features and operabilities will be apparent to those of ordinary skill in the art in view of the teachings herein.

In embodiments, the ultrasonic waveguide may be flexible, semi-flexible, rigid, or have any other suitable properties. As noted above, ultrasonic transducer 122 may be integrally coupled with the ultrasonic blade 130 via ultrasonic waveguide. In embodiments, when ultrasonic transducer 122 is activated to vibrate at ultrasonic frequencies, such vibrations are communicated through ultrasonic waveguide to ultrasonic blade 130, such that the ultrasonic blade 130 will also vibrate at ultrasonic frequencies. When the ultrasonic blade 130 is in an activated state, such that the blade 130 is vibrating ultrasonically, the ultrasonic blade 130 is operable to effectively cut through bone matter or other desired tissue. In embodiments, the ultrasonic transducer 122, ultrasonic waveguide, and ultrasonic blade 130 together may form an acoustic assembly providing ultrasonic energy for surgical procedures when powered by a power source. In embodiments, the hand assembly 102 may be configured to substantially isolate the operator from the vibrations of the acoustic assembly formed by the ultrasonic transducer 122, ultrasonic waveguide, and ultrasonic blade 130.

In embodiments, the one or more control switches 104 may be associated with a “activation” mode, such that actuating a particular one of the control switches 104 initiates ultrasonic vibrations, through the ultrasonic blade 134, either by continuously holding down the control switch 104 or by pressing and releasing the control switch 104 to activate and deactivate the ultrasonic vibrations. In embodiments, activation of one of the control switches 104 may cause vibration of ultrasonic blade 130 at a relatively low amplitude. In embodiments, activation of the same or alternative control switches may cause vibration of ultrasonic blade 130 at a relatively high amplitude. Other suitable operational modes that may be associated with the one or more control switches 104 will be apparent to persons skilled in the art in view of the teachings herein.

In embodiments, it may be useful to detach the transmission assembly 110 from the handle assembly 102 and transducer assembly 120. For instance, a detachable transmission assembly 110 may permit the reuse of the handle assembly 102 with multiple transmission assemblies 120 having various end effectors 130. In embodiments, the ultrasonic blade 130 may have different sizes, shapes, and features embedded into the ultrasonic blade 130, as described herein. Furthermore, a handle assembly 102 may be reused for different operations by a user, such that a dirty transmission assembly 110 may be removed, optionally cleaning the handle assembly 102, and coupling a new transmission assembly to the handle assembly 102 for a new operation. Accordingly, in embodiments, enabling the handle assembly 102 to be coupled with a variety of transmission assemblies 110, which may be preferable for some users of the surgical instrument 100. In embodiments, the transducer assembly 120 may be reused for different operations by a user, such that hand assembly 102 and the transmission assembly 110 may be removed and a new handle assembly 102 or new transmission assembly 110 integrated for a new operation. In embodiments, the transducer assembly 120 may be readily attachable and detachable to the hands assembly 102 and the transmission assembly 110, such that the transducer assembly may be detached and reused. Accordingly, in embodiments, enabling the transuder assembly 120 to be coupled with a variety of hand assemblies 102 and transmission assemblies 110, which may be preferable for some users of the surgical instrument 100.

In embodiments, the ultrasonic surgical instrument 100 and various features as described herein may be used manually in open operations, laparoscopically, or in conjunction with robotic systems, like the Hugo RAS and Mazor X.

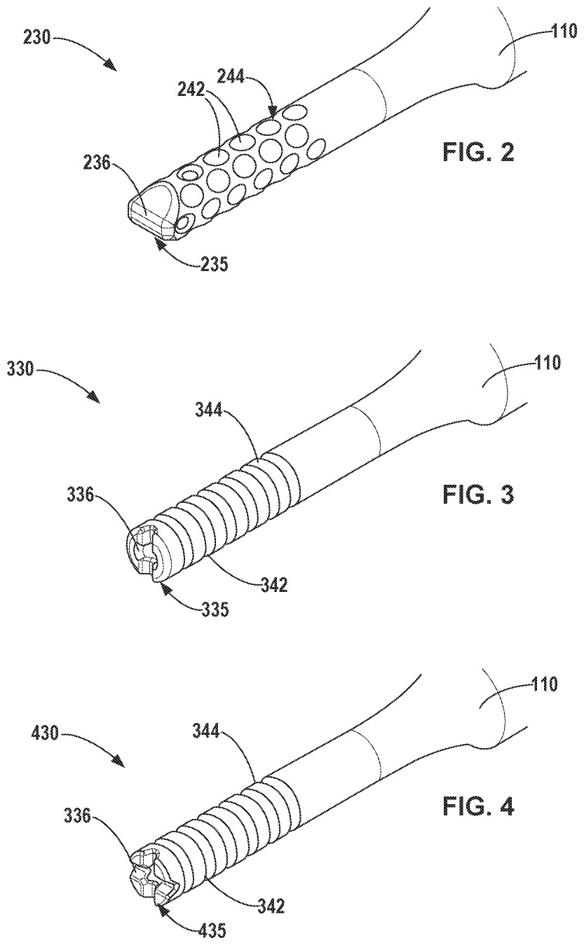

With additional reference to FIGS. 2-8B, FIGS. 2-8B illustrates a series of features for a variety of ultrasonic blades enabled to be integrated and otherwise used with an ultrasonic surgical instrument. In embodiments, among at least a portion of the ultrasonic blade 130, integration of a series of features or embodiments, either separately or in combination, may be embedded and adapted into the structure of the ultrasonic blade 130. The variety of ultrasonic blades, as described herein, may be enabled with an ultrasonic surgical device, such as the ultrasonic surgical device 100, and integrated with or receivable by transmission assembly 110 at least as part of the ultrasonic blade 130. In embodiments, the ultrasonic blade 130 may comprise one of the ultrasonic blades and may mechanically and acoustically be coupled to the waveguide and transmission assembly 110 to execute, in part, ultrasonic vibrations during surgical procedures, which may use kinetic energy and pressure to heat, fracture, or pulverize dense tissues, such as bone, as described herein.

FIG. 2 illustrates a perspective view of a dimpled pattern ultrasonic blade, according to embodiments. In embodiments, an ultrasonic blade 230 may be circular in nature and comprise a plurality of dimples 242 stretching along and around the exterior circumference of the distal portion of the ultrasonic blade 230. The plurality of dimples 242 may be embedded, creating empty indentations, into the surface of the ultrasonic blade 230, such that the dimples 242 create an offset pattern, as illustrated. In embodiments, the plurality of embedded dimples 242 each create a plurality of edges 244, in this case a plurality of circular edges, such that the edges 244 form an abrasive surface. In embodiments, the abrasive surfaces enable the breaking-up or dissection of tissue, for example, by cutting, fracturing, etc. of the dense tissue when in operation, particularly when not operating on a pure vertical axis, in part, as the plurality of edges 244 drag and vibrate across the dense tissue. In embodiments, a “pure vertical axis” may include being parallel to the long axis of the transmission assembly 110 and blade 130. In embodiments, the embedded dimples may provide an area or channel-like depressions in which the byproducts, such as the resultants from the operation of the device, and excess fluids like blood, tissue, bone fragments, etc., may be collected. In embodiments, the area or channel-like depressions provide areas and edges that may enable the expelling of byproducts and may prevent the byproduct from interfering with the working surface of the patient. In embodiments, the area or channel-like depressions act similar to flutes on a drill bit, which the dimples may scrap and expel material as the surgical device vibrates. It should be understood that while a circular dimple pattern is illustrated, alternative shapes and dimensions, such as triangles, squares, hexagons, etc. may be implemented and is not limited thereto. In embodiments, the blade tip 235 may be of a dull wedge-shaped construction. In embodiments, the wedge-shaped blade tip 235 may enable highly effective dense tissue cutting at both pure vertical and non-vertical axial operations. In embodiments, one or more lumens may or may not run the entire length of the blade and exit through just above or below the wedge in one or more locations as irrigation channels, as described herein. In embodiments, one or more lumens may exit through the center of the wedge.

FIG. 3 illustrates a perspective view of a radially grooved pattern ultrasonic blade, according to embodiments. In embodiments, ultrasonic blade 330 may comprise an alternative ultrasonic blade configuration. In embodiments, an ultrasonic blade 330 may be circular in nature and comprise a plurality of radial grooves 342 stretching along and around the exterior circumference of the distal portion of the ultrasonic blade 330. The plurality of radial grooves 342 may be embedded, creating empty channels, into the surface of the ultrasonic blade 330, such that the radial grooves 342 create an offset pattern, similarly depicted. In embodiments, the plurality of embedded radial grooves 342 each create a plurality of edges 344 (in this case a plurality of perpendicular edges relative to the length of the ultrasonic blade 330), such that the edges 344 form an abrasive surface. In embodiments, the abrasive surfaces enable the breaking-up or dissection of tissue, for example, by cutting, fracturing, etc. of the dense tissue when in operation, particularly when operating at a non-pure vertical axis. In part, as the plurality of edges 344 drag and vibrate across the dense tissue. It should be understood that while an evenly spaced radial pattern is illustrated, alternative sizes, dimensions, spatial distances, etc. may be implemented and is not limited thereto. In embodiments, the blade tip 335 may be of an arch-shaped construction, wherein the arch-shape enables grooves for irrigation control and displacement.

In embodiments, the arch-shaped blade tip 335, while potentially enabling slightly less effectiveness for dense tissue cutting as the wedge, particularly when approaching near a vertical operational direction, enables slightly higher protection against soft tissue damage. In embodiments, a single, central lumen may run the entire length of the blade and exit out the tip surface 336 for irrigation, as described herein. In embodiments, two irrigation channels may extend outward radially from the central irrigation lumen, creating two arcing crenelations on the tip surface 336. In embodiments, any number of irrigation channels may extend outward from the central irrigation channel creating any number of crenelations on the tip surface 336. In other embodiments, no irrigation channels may extend outwardly, thus forming a continuous ring at the tip surface 336 of the blade tip 335.

FIG. 4 illustrates a perspective view of a grooved pattern ultrasonic blade, similarly depicted in FIG. 3, with a crenelated blade tip, according to embodiments. In embodiments, ultrasonic blade 430 may comprise an alternative blade tip configuration. The blade tip 435 may form a crenelated blade tip pattern, wherein the crenelated blade tip enables additional or alternative grooves for irrigation control and displacement in comparison to the arch-shaped construction of FIG. 3. The crenelated blade tip 435, while potentially enabling slightly less effectiveness for dense tissue cutting, due to less contact exposure of the blade as compared to the arch-shape, enables greater protection against soft tissues damage.

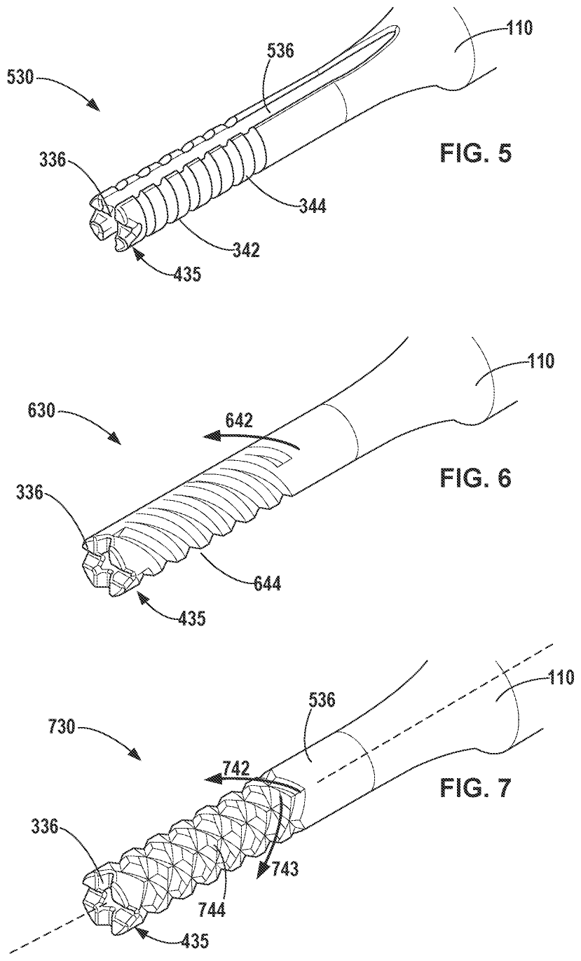

FIG. 5 illustrates a perspective view of a grooved pattern ultrasonic blade, similarly depicted in FIG. 4, with a cross-shaped blade tip and dual lumen irrigation grooves, according to embodiments. In embodiments, ultrasonic blade 530 may comprise an alternative lumen configuration. In embodiments, the ultrasonic blade 530 may comprise two lumens 536 configured to run internally through the transmission assembly 110, through the proximal portion of the blade, and become external at the distal end of an ultrasonic blade 530. While a lumen 336 may be depicted above and a similar lumen below the ultrasonic blade 530, establishing dual axial lumens with 180° separation, the lumens may be configured to become external at alternative locations and alternative degrees of separation, such as 25°, 45°, 90°, etc. While the lumens 336 may potentially enable slightly less effectiveness for dense tissue cutting, the additional lumens and exposure may provide additional irrigation, as compared to other ultrasonic blade features.

FIG. 6 illustrates a perspective view of a spirally grooved pattern ultrasonic blade, according to embodiments. In embodiments, ultrasonic blade 630 may comprise an alternative ultrasonic blade configuration. In embodiments, an ultrasonic blade 630 may be circular in nature and comprise a singular, radial groove 642 stretching helically along and around the exterior circumference of the distal portion of the ultrasonic blade 630. It should be understood that while a singular, radial groove 642 is illustrated, additional grooves, alternative sizes, dimensions, spatial distances, etc. may be implemented and is not limited thereto. In embodiments, one or more radial groove(s) 642 may be integrated such that the grooves stretch helically along and around the exterior circumference of the distal portion of the ultrasonic blade 630. In embodiments, the helical groove(s) 642 may be embedded, creating empty channels, into the surface of the ultrasonic blade 630, such that the helical groove(s) 642 creates helical edges 644, in this case a plurality of continuous edges relative to the length of the ultrasonic blade 630, such that the edges 644 form an abrasive surface. In embodiments, the abrasive surfaces enable the breaking-up or dissection of tissue, for example, by cutting, fracturing, etc. of the dense tissue when in operation, particularly when operating at a non-pure vertical axis, in part, as the plurality of edges 644 drag and vibrate across the dense tissue. In embodiments, the helical groove(s) 642 may act similar to flutes on a drill bit, which the dimples may scrap and expel material as the surgical device vibrates. It should be understood that while an evenly spaced helical pattern is illustrated, alternative sizes, dimensions, spatial distances, etc. may be implemented, these features are illustrated by way of example, and not of limitation. In embodiments, the helical groove(s) 642 may cause the ultrasonic blade 630 to twist, relative to the handle assembly (not depicted) or the transmission assembly 110 as the ultrasonic blade 630 extends and retracts ultrasonically, which may increase the potential cutting power.

In embodiments, the blade tip 435 may form a cross-shaped pattern, as described herein. In embodiments, a single, central lumen may run the entire length of the blade and exit out the tip 336 for irrigation, as described herein. In embodiments one or more lumens may or may not run the entire length of the ultrasonic blade 330 and exit through alternative locations of the ultrasonic blade tip 435.

FIG. 7 illustrates a perspective view of a diamond grooved pattern ultrasonic blade, similarly depicted in FIG. 6, with a cross-shaped blade tip, according to embodiments. In embodiments, ultrasonic blade 730 may comprise an alternative ultrasonic blade configuration. In embodiments, an ultrasonic blade 730 may be circular in nature and comprise a first radial groove 742 stretching helically along and around the exterior circumference of the distal portion of the ultrasonic blade 730, in a first counterclockwise direction. The helical groove 742 may be embedded, creating empty channels, into the surface of the ultrasonic blade 730, such that the helical groove 742 creates helical edges 744, in this case a plurality of continuous edges relative to the length of the ultrasonic blade 730, such that the edges 744 form an abrasive surface.

In embodiments, the ultrasonic blade 730 may also comprise a second radial groove 743 stretching helically along and around the exterior circumference of the distal portion of the ultrasonic blade 730, in a second clockwise direction. The helical groove 43 may be embedded, creating empty channels, into the surface of the ultrasonic blade 730, such that the helical groove 742 creates helical additional edges 744, in this case a second plurality of continuous edges relative to the length of the ultrasonic blade 730, such that the edges 744 form an abrasive surface. The two opposing radial grooves 742 and 743 intersect, creating crossed spiral grooves along the ultrasonic blade to form a diamond pattern. In embodiments, the helical grooves 742 and 743 may be embedded into the surface of the ultrasonic blade 730, such that the helical grooves 742 and 743 create a plurality of diamond-shaped edges 744 (i.e., from the protruding diamond-shaped edges), such that the edges 744 form an abrasive surface. In embodiments, the embedded surfaces enable the breaking-up or dissection of tissue, for example, by cutting, fracturing, etc. of the dense tissue when in operation, particularly when not operating on a pure vertical axis, in part, as the plurality of edges 744 drag and vibrate across the dense tissue.

It should be understood that while an evenly spaced helical patterns and a diamond shape is illustrated, alternative sizes, dimensions, spatial distances, etc. may be implemented, these features are illustrated by way of example, and not of limitation. In embodiments, the blade tip 435 may be of cross-shaped pattern, as described herein. In embodiments, a single, central lumen may run the entire length of the blade and exit out the tip 336 for irrigation, as described herein. In embodiments one or more lumens may or may not run the entire length of the ultrasonic blade 730 and exit through alternative locations of the ultrasonic blade tip 435. While the multiple grooves 742 and 743 may potentially enable slightly less effectiveness for dense tissue cutting, due a more balanced intensity of exposure of tissue to the abrasive surface edges 744, the balanced exposure enables slightly higher protection against soft tissue damage, as compared to other ultrasonic blade features.

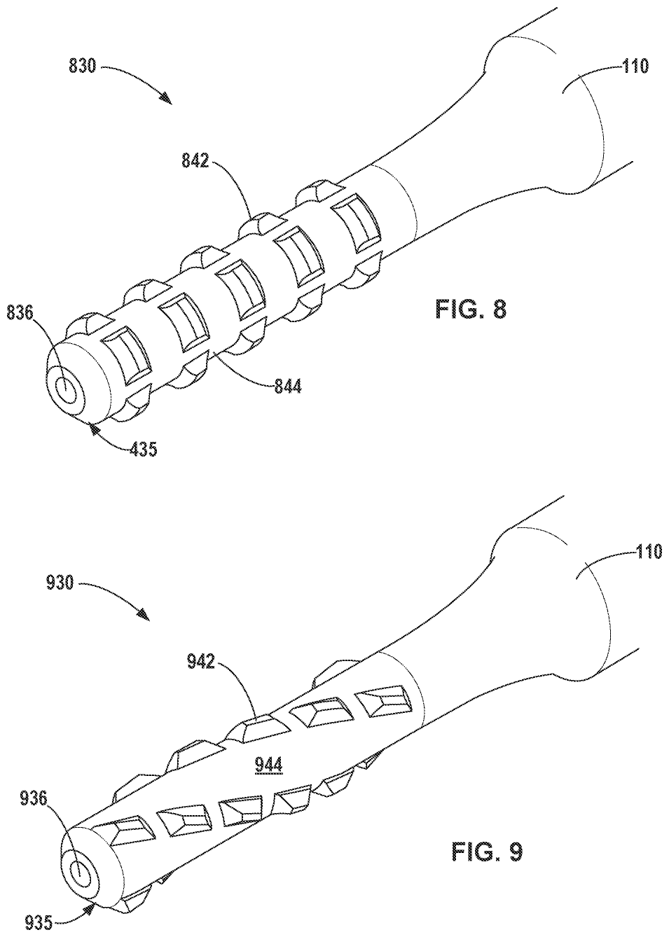

FIG. 8 illustrates a perspective view of an ultrasonic blade, according to embodiments. In alternative embodiments, ultrasonic blade 830 may be circular in nature and comprise a plurality of radial protrusions 842 stretching along and around the exterior circumference of the distal portion of the ultrasonic blade 830. The plurality of radial protrusions 842 may extend outward from the surface of the ultrasonic blade 830, which may create channels 844, along the surface of the ultrasonic blade 830. In embodiments, the radial protrusions 842 may create an offset pattern, similarly depicted. In embodiments, the plurality of radial protrusions 842 each create a plurality of edges (in this case a plurality of perpendicular edges relative to the length of the ultrasonic blade 830), such that the edges form abrasive surfaces. In embodiments, the abrasive surfaces enable the breaking-up or dissection of tissue, for example, by cutting, fracturing, etc. of the dense tissue when in operation, particularly when operating at a non-pure vertical axis, in part, as the plurality of edges drag and vibrate across the dense tissue. In embodiments, the channels 844 may act similar to flutes on a drill bit, which may scrap and expel material as the surgical device vibrates. It should be understood that while an evenly spaced radial protrusion pattern is illustrated, alternative sizes, dimensions, spatial distances, etc. may be implemented and is not limited thereto. In embodiments, the blade tip 835 may be of an oval or circular shaped construction.

In embodiments, a single, central lumen may run the entire length of the blade and exit out the tip 836 for irrigation, as described herein. In embodiments, any number of irrigation channels may extend outward from the central irrigation channel creating any number of crenelations on the tip surface 836. In other embodiments, no irrigation channels may extend outwardly, thus forming a continuous ring at the tip surface 836 of the blade tip 835.

FIG. 9 illustrates a perspective view of a spiral pattern ultrasonic blade, according to embodiments. In alternative embodiments, similar to FIG. 8, ultrasonic blade 930 may comprise alternative protrusion configurations. In embodiments, an ultrasonic blade 930 may be circular in nature and comprise radial protrusions 942 stretching helically along and around the exterior circumference of the distal portion of the ultrasonic blade 930. In embodiments, the protrusions 942 may be incorporated onto the ultrasonic blade 930) such that they extend outward from the surface of the ultrasonic blade 930, which may create channels 944, along the surface of the ultrasonic blade 930. In embodiments, the protrusions 942 form abrasive surfaces which enable the breaking-up or dissection of tissue, for example, by cutting, fracturing, etc. of the dense tissue when in operation, particularly when operating at a non-pure vertical axis, in part, as the surfaces drag and vibrate across the dense tissue. In embodiments, the channels 944 may act similar to flutes on a drill bit, which may scrap and expel material as the surgical device vibrates. It should be understood that while an evenly spaced protrusion pattern is illustrated, alternative sizes, dimensions, spatial distances, etc. may be implemented, these features are illustrated by way of example, and not of limitation. In embodiments, the protrusions 942 may cause the ultrasonic blade 930 to twist, relative to the handle assembly (not depicted) or the transmission assembly 110 as the ultrasonic blade 930 extends and retracts ultrasonically, which may increase the potential cutting power.

In embodiments, the blade tip 935 may be a blade tip as described herein. In embodiments, a single, central lumen may run the entire length of the blade and exit out the tip 936 for irrigation, as described herein. In embodiments one or more lumens may or may not run the entire length of the ultrasonic blade 930 and exit through alternative locations of the ultrasonic blade tip 935.

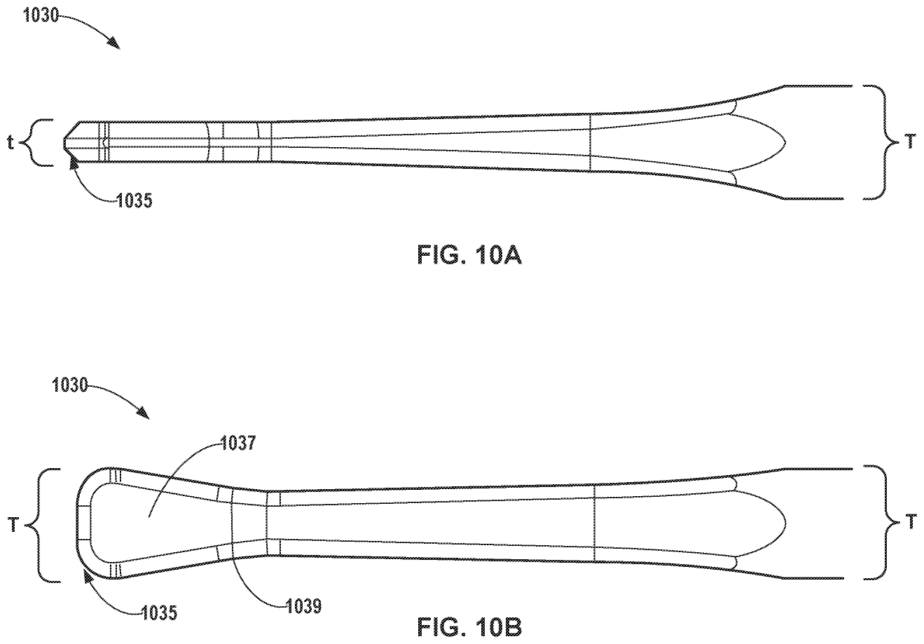

FIGS. 10A-B illustrate a side view and a side view of a scalpel-type ultrasonic blade, according to embodiments. In embodiments, ultrasonic blade 1030 may comprise an alternative ultrasonic blade configuration. In embodiments, referring to FIG. 10A, an ultrasonic blade 1030 may be semi-rectangular in construction with a high energy tip 1035. The high energy tip 1035 may be highly effective for cutting, pulverizing, etc. exceedingly dense and more resistant tissue, such as cortical bone due to the concentrated edge. In embodiments, the distal end of the ultrasonic blade 1030 may be relatively thin compared to the proximal end coupled to the transmission assembly 110. In embodiments, the distal portion of the ultrasonic blade 1030 has a thickness t and the proximal portion has a thickness T, wherein t<T. In embodiments, the thickness of the ultrasonic blade 1030 may gradually increase as the distal end approaches the proximal end. In embodiments, thickness t may be between a range of 0.010 inches to 0.050 inches. In embodiments, thickness T may be between a range of 0.080 inches to 0.200 inches. While the ultrasonic blade may be configured to include a variety of thickness ranges for bot t and T, these features are illustrated by way of example, and not of limitation.

Referring to FIG. 10B, FIG. 10B illustrates a top view of the scalpel-type ultrasonic blade. In embodiments, the distal end of the ultrasonic blade 1030, as seen by a 90° rotation, may be configured to have a flat portion 1037. In embodiments, the flat portion 1037 may have a smooth surface. In embodiments, the flat portion 1037 may have a textured surface, such as dimples, grooves, or cross hatching. In embodiments, the high energy tip 1035 may be, at least in part, partially rounded off at the foremost distal edge of the ultrasonic blade 1030. In embodiments, the high energy tip 1035 and extending thin cutting surface may up to or beyond recurve 1039, at least in part, include a textured surface such as dimples, grooves, or cross hatching to form a serration. In embodiments, the distal end of the ultrasonic blade 1030 may begin to decrease in width prior to approaching the recurved edge 1039. From the recurved edge 1039, the ultrasonic blade 1030 may then begin to gradually increase in width as the foremost proximal end approaches, thus be coupled to the transmission assembly 110 to execute, at least in part, ultrasonic vibrations during surgical procedures. The thin, smooth recurved edge configured by way of the thin structure (illustrated in FIG. 10A) and the flat surface 1037, in combination with the recurved structure 1039 (illustrated in FIG. 10B), decreases the change in velocity across the blade and creates a more uniform cutting surface. The recurve feature 1039 creates a pocket when dragging the blade that provides greater haptic feedback to effectively differentiate between dense tissue (e.g., bone) and other softer tissue. In embodiments, the flat portion enables the sealing and coagulation of smaller vessel and ducts. It should be understood that while a particular scalpel-type blade is illustrated, alternative sizes, dimensions, spatial distances, etc. may be implemented, these features are illustrated by way of example, and not of limitation.

As described herein, ultrasonic devices with the features described herein may improve the ability of a user, such as a surgeon, to remove dense tissue like bone or other tough tissues more quickly and safely without or with minimal damage to adjacent soft or flexible tissues. In embodiments, a single or combination of these features integrated along an ultrasonic blade, described herein, may aid in the abrading (horizontal pull) of dense tissue, wherein the corresponding edges from the embedded patterns may enable the expelling of byproducts from the working surface of the patient and may prevent the byproducts from interfering with the procedure. In embodiments, the area or channel-like depressions act similar to flutes on a drill bit, which the dimples may scrap and expel material as the surgical device vibrates.

In embodiments, the various tip features may provide additional aid in drilling (vertical push) and act to concentrate the overall pressure exerted by the ultrasonic blade. In embodiments, the various tip features may provide additional aid in protection to soft tissues. In embodiments, the surfaces of the various blade tips may be configured to intentionally not be sharp or have acute angles, which may reduce the potential risk of cutting or otherwise negatively affecting non-intended tissues like arteries, connective tissue, etc., referred to as elastic tissue.

In embodiments, the various lumens or irrigation channels may be integrated such that fluids, medication, or other substance beneficial for operations of an ultrasonic surgical procedure, such as saline to clear or clean out the working area, sterilization fluid, etc., may be irrigated to the working surface of the patient during operation. In embodiments, the various suction channels or lumens may be integrated through an ultrasonic blade to suction byproducts, such as those resulting from operation of the device and excess fluids, such as blood, tissue, bone fragments, etc., from the working surface of the patient. In embodiments, multiple lumens or channels may be integrated into the ultrasonic surgical instrument 100 and the ultrasonic blade such that electrosurgical operations, suction and irrigation may be performed simultaneously or individually.

In embodiments, the features integrated into the ultrasonic blade may have an overall effect similar to an end mill but may proceed more like a grinder or file, differentiating from an actual cutting motion. In embodiments, the abrasive surfaces, as described herein, may be intentionally integrated as to not be too deeply embedded or have acute angles, relative to the ultrasonic blade and the waveguide, thus reducing the risk of cutting or otherwise negatively affecting non-intended tissues, such as elastic tissue. In embodiments, embedding the portions of the ultrasonic blade deeper, which would result in more intense abrasive surfaces, may provide an increase in cutting ability and vice versa.

While varying patterns, edges and otherwise ultrasonic blade shapes are illustrated, these features are illustrated by way of example, and not of limitation. It should be understood that varying ultrasonic blade lengths, blade circumferences, pattern edge depths, edge sharpness, waveguide patterns, etc. may be implemented without deviating from scope of the present disclosure. Furthermore, additional features may be included to or removed from the ultrasonic blade. In embodiments, the ultrasonic blade itself may be constructed in a variety of shapes and sizes. While ultrasonic blades have been depicted to have a cylindrical shape or semi-rectangular shape, these features are illustrated by way of example, and not of limitation. It should be understood that the ultrasonic blade may be cylindrical, triangular, polyhedral, hemi-cylindrical, square, hooked, and/or any other shape and include one or of the features described herein. Furthermore, additional features may be added to the tip of the ultrasonic blade, including spherical tips, hooked tips, square tips, serrated edging, and any other additional features and variations as similarly described herein.

In embodiments, the ultrasonic blades may be readily detachable from and attachable to the surgical instrument 100, via the transmission assembly 110, which would enable different patterns, edges, etc., as illustrated herein, to be utilized. For example, ultrasonic blades that cut dense tissue more efficiently may be easily swapped for an ultrasonic blade that more effectively protects soft tissue. Depending on circumstances of the procedure, a user may initially require a higher energy intensity ultrasonic blade, referring to FIGS. 10A-B, during the initial stages of the procedure but may later require a less intensive ultrasonic blade, for example, refereeing to FIG. 2. For example, during the operation of a procedure, the user may have reached areas where softer tissue is more of a concern, in which an alternative ultrasonic blade with differing features may be desirable. In another example, a user may require an alternative ultrasonic blade following a previous procedure, in which case, a current ultrasonic blade may be quickly and easily swapped. Thus, in embodiments, any variation of ultrasonic blades, and variation thereof, may be coupled with a variety of transmission assemblies 110.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity; it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(1) are not to be invoked unless the specific terms “means for” or “step for” are recited in a claim

What is claimed is:

1. A surgical instrument, comprising:
an ultrasonic transducer contained in a housing;
a transmission assembly, at least partially received by the housing and encapsulating a waveguide, wherein the waveguide is configured to transmit ultrasonic vibrations from the transducer to the ultrasonic blade;
the ultrasonic blade including a blade tip and at least one incorporated feature between a distal end of the ultrasonic blade and a proximal end, wherein the at least one incorporated feature includes one or more depressions or protrusions, forming abrasive surfaces and channels, wherein the abrasive surfaces breakup tissue and the channels expel the broken-up tissue; and
a control switch operable to initiate and halt the ultrasonic transducer.

2. The surgical instrument of claim 1, wherein the at least one incorporated feature includes an offset pattern of embedded dimples, wherein the edges of the dimples have abrasive surfaces.

3. The surgical instrument of claim 1, wherein the at least one incorporated feature includes one or more perpendicular radial grooves stretching along and around the circumference of the ultrasonic blade, wherein the edges of the grooves have abrasive surfaces.

4. The surgical instrument of claim 1, wherein the at least one incorporated feature includes one or more radial grooves stretching helically along and around the circumference of the ultrasonic blade, wherein the edges of the grooves have abrasive surfaces.

5. The surgical instrument of claim 1, wherein the at least one incorporated feature includes a first radial groove stretching helically along and around the circumference of the ultrasonic blade in a first direction; and a second radial groove stretching helically along and around the circumference of the ultrasonic blade in a second direction, wherein the first and second radial groves intersect and create crossed spiral grooves, wherein the edges of the grooves have abrasive surfaces.

6. The surgical instrument of claim 1, wherein the blade tip includes at least one of wedge-shaped, arc-shaped, ring, cross-shaped, or circular-shaped.

7. The surgical instrument of claim 1, wherein the distal end is configured to have a recurve configuration the distal end: the distal end of the blade tip being relatively thin compared to the proximal end; and the blade tip including a smooth, flat surface.

8. The surgical instrument of claim 1, wherein the ultrasonic blade includes one or more lumens to irrigate out of the blade tip.

9. The surgical instrument of claim 8, wherein the ultrasonic vibration transmission and irrigation out of the blade tip is performed simultaneously.

11. The surgical instrument of claim 1, wherein the ultrasonic blade is readily attachable to and detachable from the transmission assembly.

12. An ultrasonic surgical tip, comprising:
an ultrasonic blade including a blade tip at a distal end configured to be coupled to a waveguide, encapsulated by a transmission assembly, at a proximal end, wherein the waveguide is configured transmit ultrasonic vibrations from the transducer to the ultrasonic blade; and
the ultrasonic blade including at least one incorporated feature, at least in part, stretching between the distal end and the proximal end, wherein the at least one incorporated feature includes one or more depressions or protrusions, forming abrasive surfaces and channels, wherein the abrasive surfaces breakup tissue and the channels expel the broken-up tissue.

13. The ultrasonic surgical tip of claim 11, wherein the at least one incorporated feature includes an offset pattern of embedded dimples, wherein the edges of the dimples have abrasive surfaces.

14. The ultrasonic surgical tip of claim 11, wherein the at least one incorporated feature includes one or more perpendicular radial grooves stretching along and around the circumference of the ultrasonic blade, wherein the edges of the grooves have abrasive surfaces.

15. The ultrasonic surgical tip of claim 11, wherein the at least one incorporated feature includes a single, radial groove stretching helically along and around the circumference of the ultrasonic blade, wherein the edges of the grooves have abrasive surfaces.

16. The ultrasonic surgical tip of claim 11, wherein the at least one incorporated feature includes a first radial groove stretching helically along and around the circumference of the ultrasonic blade in a first direction; and a second radial groove stretching helically along and around the circumference of the ultrasonic blade in a second direction, wherein the first and second radial groves intersect and create crossed spiral grooves, wherein the edges of the grooves have abrasive surfaces.

17. The ultrasonic surgical tip of claim 11, wherein the blade tip includes at least one of wedge-shaped, arc-shaped, ring, cross-shaped, or circular-shaped.

18. The ultrasonic surgical tip of claim 11, wherein the ultrasonic blade includes one or more lumens to irrigate out of the blade tip.

19. The ultrasonic surgical tip of claim 18, wherein the ultrasonic vibration transmission and irrigation out of the blade tip is performed simultaneously.

20. The ultrasonic surgical tip of claim 1, wherein the ultrasonic blade is readily attachable to and detachable from the transmission assembly.
